(19) United States
(12) Patent Application Publication
(10) Pub. No.: US 2026/0083850 A1
(43) Pub. Date: Mar. 26, 2026

(54) AGENTS AND METHODS FOR RADIATION DOSE ENHANCEMENT OR TUMOR DELINEATION

(71) Applicant: University of Connecticut, Farmington, CT (US)

(72) Inventors: Henry M. Smilowitz, West Hartford, CT (US); James Hainfeld, Shoreham, NY (US)

(73) Assignee: University of Connecticut, Farmington, CT (US)

(21) Appl. No.: 19/338,756

(22) Filed: Sep. 24, 2025

Related U.S. Application Data
(60) Provisional application No. 63/698,602, filed on Sep. 25, 2024.

(51) Int. Cl. A61K 41/00; A61K 33/00; A61N 5/10; A61P 35/00

(52) U.S. Cl. CPC A61K 41/0038; A61K 33/00; A61N 5/10; A61P 35/00; A61N 2005/1098

(57) ABSTRACT

The disclosure is directed to compounds, compositions, and methods for radiation dose enhancement and the delineation of tumors, and in particular, to improve dose of megavoltage radiation therapy using barium nanoparticles such as BaTiO₃ nanoparticles for the delineation of tumors.

### RELATED APPLICATIONS

The instant application claims priority to U.S. Provisional Application No. 63/698,602, filed on Sep. 25, 2024. The entire contents of each of the foregoing applications are expressly incorporated by reference herein in their entirety.

### FIELD OF THE DISCLOSURE

The present disclosure relates to agents, compositions, and methods for radiation dose enhancement and/or delineation of tumors, and in particular, the agents, compositions, and methods including barium nanoparticles (Ba-NPs) such as BaTiO₃ nanoparticles (NPs) to improve radiation therapy (RT) and ultrasound therapy and to better delineate tumors.

### BACKGROUND

Various forms of radiation such as X-rays, laser light, and microwaves, as well as particle beams of, for example, neutrons, electrons, and protons, have been used to treat tumors. However, such radiation are not generally very specific for the tumor, and the dosages used often result in serious damage to normal tissue, thus limiting irradiation to lower doses that are less effective. Thus, there is an immediate need for novel compounds and methods that can be safely used at levels suitable for tumor radiation dose enhancement.

### SUMMARY

The following brief summary is not intended to include all features and aspects of the present disclosure, nor does it imply that any claim must include all features and aspects discussed in this summary.

Accordingly, in one aspect, the present invention provides a method of enhancing effects of radiation directed to a tissue or a population of cells of a subject in need thereof, comprising: administering an effective amount of barium nanoparticles to the subject, such that said barium nanoparticles are brought into the vicinity of or into contact with said tissue or a population of cells; and subjecting the subject to radiation under the presence of said barium nanoparticles.

In one embodiment, the subject is mammal. In one embodiment, the mammal is a human. In one embodiment, the said tissue or said population of cells is tumor.

In one embodiment, said tumor is a solid tumor. In one embodiment, the solid tumor is selected from the group comprising of prostate cancer, pancreatic cancer, lung cancer, breast cancer, colon cancer, carcinoma, melanoma, sarcomas, lymphoma, or any combination thereof.

In one embodiment, the barium nanoparticles are BaTiO₃ nanoparticles. In one embodiment, the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

In one embodiment, the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In one embodiment, the barium nanoparticles are administered with intravenous, or intratumoral injection.

In one embodiment, the barium nanoparticles are administered with intravenous rejection. In one embodiment, effective amount of barium nanoparticles administered with intravenous rejection per kilogram of the tumor is about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g.

In one embodiment, the barium nanoparticles are administered with intratumoral injection. In one embodiment, the effective amount of barium nanoparticles administered with intratumoral injection per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

In one embodiment, the radiation is X-ray, laser light, microwave, particle beam, or gamma ray. In one embodiment, wherein the radiation is X-ray. In one embodiment, the radiation is microwave. In one embodiment, the particle beam is proton beam.

In one embodiment, the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 keV to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 MeV.

In one embodiment, the barium nanoparticles are administered with a concentration of at least about 0.1% metal by weight of the subject. In one embodiment, the barium nanoparticles are administered with a concentration of may be less than 0.1% metal by weight of the subject. In one embodiment, the barium nanoparticles are administered with a concentration of up to about 10% metal by weight of the subject.

In one embodiment, the method further comprises administering one or more additional radiation enhancer or sensitizer. In one embodiment, the additional radiation enhancer or sensitize is Fluorouracil.

In another aspect, the present invention provides a method for delineation of tumors, the method comprising: administering an effective amount of contrast agent to a subject in need thereof; imaging the subject with an imaging technique to detect a signal from the contrast agent; and delineating boundaries of the tumor based on a detected signal from the contrast agent; wherein the contrast agent is barium nanoparticle.

In one embodiment, the subject is mammal. In one embodiment, the mammal is a human.

In one embodiment, the tumor is a solid tumor selected from the group comprising of prostate cancer, pancreatic cancer, lung cancer, breast cancer, colon cancer, carcinoma, melanoma, sarcomas, lymphoma, or any combination thereof.

In one embodiment, the barium nanoparticles are BaTiO₃ nanoparticle. In one embodiment, the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

In one embodiment, the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In one embodiment, the barium nanoparticles are administered with intravenous or intratumoral injection.

In one embodiment, the barium nanoparticles are administered with intravenous injection. In one embodiment, the effective amount of barium nanoparticles administered with intravenous injection per kilogram of the tumor is about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g.

In one embodiment, the barium nanoparticles are administered with intratumoral injection. In one embodiment, the effective amount of barium nanoparticles administered with intratumoral injection per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, or about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

In one embodiment, the barium nanoparticles are administered with a concentration of at least about 0.1% metal by weight of the subject. In one embodiment, the barium nanoparticles are administered with a concentration of may be less than 0.1% metal by weight of the subject. In one embodiment, the barium nanoparticles are administered with a concentration of up to about 10% metal by weight of the subject.

In one embodiment, the imaging technique is selected from the group comprising: MicroCT, clinical CT, planar X-ray, mammography unit, or darkfield microscopy.

In one embodiment, the energy source of the imaging technique is X-ray. In one embodiment, the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV. In one embodiment, the X-ray is about 20 keV to about 40 keV, about 40 keV to about 60 keV, or about 60 keV to about 80 keV. In one embodiment, the X-ray is about 60 keV.

In another aspect, the present invention provides a method of treating cancer in a subject in need thereof, the method comprising: administering an effective amount of barium nanoparticles or a composition including barium nanoparticles to the subject; and administering radiotherapy to the said subject.

In one embodiment, the subject is mammal. In one embodiment, the mammal is a human. In one embodiment, the cancer is selected from the group comprising of prostate cancer, pancreatic cancer, lung cancer, breast cancer, colon cancer, carcinoma, melanoma, sarcomas, lymphoma, and any combination thereof.

In one embodiment, the barium nanoparticles comprise BaTiO₃ nanoparticles. In one embodiment, the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

In one embodiment, barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In one embodiment, the barium nanoparticles are administered with intravenous or intratumoral injection.

In one embodiment, the barium nanoparticles are administered with intravenous injection. In one embodiment, the effective amount of barium nanoparticles administered with intravenous injection per kilogram of the tumor is about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g.

In one embodiment, wherein the barium nanoparticles are administered with intratumoral injection. In one embodiment, the effective amount of barium nanoparticles administered with intratumoral injection per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

In one embodiment, radiation of the radiotherapy is X-ray, laser light, microwave, particle beam, or gamma ray. In one embodiment, the radiation is X-ray. In one embodiment, the radiation is microwave. In one embodiment, the particle beam is proton beam. In one embodiment, the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 keV to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 Me V.

In one embodiment, the method enhances or improves the ablation of cancerous tissues in the subject without unacceptable damage to surrounding normal tissues or substantial toxicity to the subject.

In one embodiment, the barium nanoparticles is administered with a concentration of at least about 0.1% metal by weight of the subject. In one embodiment, the barium nanoparticles are administered with a concentration of may be less than 0.1% metal by weight of the subject. In one embodiment, the barium nanoparticles are administered with a concentration of up to about 10% metal by weight of the subject.

In one embodiment, the method further comprises administering one or more additional radiation enhancer or sensitizer. In one embodiment, the additional radiation enhancer or sensitizer comprises Fluorouracil.

In another aspect, the present invention provides a pharmaceutical composition comprising: an effective amount of barium nanoparticles; and at least one pharmaceutical carrier and/or excipient.

In one embodiment, the barium nanoparticles are BaTiO₃ nanoparticles. In one embodiment, the size of the barium nanoparticles is about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

In one embodiment, the pharmaceutical composition is formulated for treatment of a subject having prostate cancer, pancreatic cancer, lung cancer, breast cancer, colon cancer, carcinoma, melanoma, sarcomas, lymphoma, or any combination thereof.

In one embodiment, the subject is a mammal. In one embodiment, the mammal is a human. In one embodiment, the pharmaceutical composition is suspensions in aqueous, non-aqueous or mixed media. In one embodiment, the pharmaceutical composition is formulated into tablets, capsules, gel capsules, liquid syrups, soft gels, suppositories, emulsions or microemulsions, or enemas. In one embodiment, the pharmaceutical carrier or excipient is a pharmaceutically acceptable solvent, suspending agent or any other pharmacologically inert vehicle for delivering one or more cargoes. In one embodiment, the pharmaceutical composition further comprises nucleic acids and/or antibody.

In one embodiment, the barium nanoparticles are administered with a concentration of at least about 0.1% metal by weight of the subject. In one embodiment, the barium nanoparticles are administered with a concentration of may be less than 0.1% metal by weight of the subject. In one embodiment, the barium nanoparticles are administered with a concentration of up to about 10% metal by weight of the subject.

In one embodiment, the pharmaceutical composition further comprises one or more additional radiation enhancer or sensitizer. In one embodiment, the additional radiation enhancer or sensitizer comprises Fluorouracil.

In another aspect, the present invention provides a method of treating cancer in a subject in need thereof, the method comprising: administering an effective amount of barium nanoparticles or a composition including barium nanoparticles to the subject; and administering a therapeutically effective amount of one or more immunotherapy agents to the said subject.

In one embodiment, the subject is mammal. In one embodiment, the mammal is a human.

In one embodiment, the cancer is selected from the group comprising of prostate cancer, pancreatic cancer, lung cancer, breast cancer, colon cancer, carcinoma, melanoma, sarcomas, lymphoma, and any combination thereof.

In one embodiment, the barium nanoparticles are BaTiO₃ nanoparticle. In one embodiment, the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

In one embodiment, the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In one embodiment, the barium nanoparticles are administered with intravenous or intratumoral injection.

In one embodiment, the barium nanoparticles are administered with intravenous injection.

In one embodiment, the effective amount of barium nanoparticles administered with intravenous injection per kilogram of the tumor is about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g.

In one embodiment, the barium nanoparticles are administered with intratumoral injection. In one embodiment, the effective amount of barium nanoparticles administered with intratumoral injection per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

In one embodiment, radiation of the radiotherapy is X-ray, laser light, microwave, particle beam, or gamma ray. In one embodiment, the radiation is X-ray. In one embodiment, the radiation is microwave. In one embodiment, the particle beam is proton beam. In one embodiment, the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 ke V to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 MeV.

In one embodiment, the method enhances or improves the ablation of cancerous tissues in the subject without unacceptable damage to surrounding normal tissues or substantial toxicity to the subject.

In one embodiment, the barium nanoparticles is administered with a concentration of at least about 0.1% metal by weight of the subject. In one embodiment, the barium nanoparticles are administered with a concentration of may be less than 0.1% metal by weight of the subject. In one embodiment, the barium nanoparticles are administered with a concentration of up to about 10% metal by weight of the subject.

In one embodiment, the method further comprises administering one or more additional radiation enhancer or sensitizer. In one embodiment, the additional radiation enhancer or sensitizer comprises Fluorouracil.

In one embodiment, the immunotherapy agent is immune checkpoint inhibitors, monoclonal antibodies, cytokines, or CAR-T cells. In one embodiment, the immune checkpoint inhibitor is PD-1 inhibitors, PD-L1 inhibitors, or CTLA-4 inhibitors.

### BRIEF DESCRIPTION OF THE DRAWINGS

FIGS. 1A-1B show side scatter (SSC) data from flow cytometry of human prostate cancer cells DU145 loaded with 0 mM (FIG. 1A) or 4 mM (FIG. 1B) BaTiO₃ Nanoparticles in vitro. FIG. 1A shows that the mean side scatter of the 0 mM BaTiO₃ samples is 22,018. FIG. 1B shows that the mean side scatter of the 4 mM BaTiO₃ sample after 6 washes is 79,690.

FIGS. 2A-2C show side scatter (SSC) data from flow cytometry of mouse pancreatic cancer cells PANC02 loaded with 0 mM (FIG. 2A), 4 mM (FIG. 2B), or 12 mM (FIG. 2C) BaTiO₃ nanoparticles in vitro. FIG. 2A shows that the mean side scatter of the 0 mM BaTiO₃ samples is 15,973. FIG. 2B shows that the mean side scatter of the 4 mM BaTiO₃ sample is 46,478. FIG. 2C shows that the mean side scatter of the 12 mM BaTiO₃ sample is 53,698.

FIGS. 3A-3C show electron microscopy images of human prostate cancer cells DU145 incubated with BaTiO₃ NPs.

FIGS. 4A-4C show electron microscopy images of PANC02 mouse pancreatic cancer cells incubated with BaTiO₃ NPs.

FIG. 5 shows results of viability assay for human prostate cancer cells incubated with BaTiO₃ 3 days after radiotherapy with 6 MV, 8 Gy megavoltage radiation.

FIG. 6 shows fractional change of cell viability of human prostate cancer cells incubated with BaTiO₃ 3 days after radiotherapy with 6 MV, 8 Gy megavoltage radiation. The p value for 2-tailed T Test was 0.0446 and the ratio of the 2-set averages is 1.613.

FIG. 7 shows weights of mice after 3.5 g/kg IV injection of BaTiO₃ NPs.

FIG. 8 shows microscopy image of PANC02 tumors loaded with BaTiO₃ in vivo.

FIGS. 9A-9C demonstrate microlocalization of BaTiO₃ in PANC02 tumors.

FIGS. 10A-10B show immunofluorescence images of cryosections of PANC02 tumors containing BaTiO₃ NPs, stained with VE Cadherin. FIG. 10A shows tissue section treated with anti-rabbit VE Cadherin followed by 488—labeled goat anti-rabbit antibody. FIG. 10B shows a tissue section treated with just 488—labeled goat anti-rabbit antibody.

FIGS. 11A-11C demonstrate colocalization of BaTiO₃ NPs and VE Cadherin (20X magnitude). FIG. 11A shows immunofluorescence image of tumoral VE Cadherin. FIG. 11B shows immunofluorescence image of BaTiO₃ NPs. FIG. 11C shows the colocalization of the fluorescence of BaTiO₃ NPs and VE Cadherin.

FIGS. 12A-12C demonstrate colocalization of BaTiO₃ NPs and VE Cadherin (40X magnitude). FIG. 12A shows immunofluorescence image of tumoral VE Cadherin. FIG. 12B shows immunofluorescence image of BaTiO₃ NPs. FIG. 12C shows the colocalization of the fluorescence of BaTiO₃ NPs and VE Cadherin.

FIGS. 13A-13D demonstrate the colocalization of BaTiO₃ NPs with CD31 and CD133. FIG. 13A shows the immunofluorescence image of nuclei. FIG. 13B shows the immunofluorescence image of BaTiO₃. FIG. 13C shows the immunofluorescence image of CD31. FIG. 13D shows the immunofluorescence image of CD133.

FIGS. 14A-14D demonstrate that BaTiO₃ nanoparticles enhance radiation therapy of subcutaneous (sc) Panc02 tumors growing in athymic nude mice. FIG. 14A shows the percent difference in tumor volume (the average of w²1/2 and 1²w/2) normalized to the original tumor volume on the day of irradiation, of the mice treated with both BaTiO₃ and RT. Each curve represent one individual mouse. FIG. 14B shows the percent difference in tumor volume (the average of w²1/2 and 1²w/2) normalized to the original tumor volume on the day of irradiation, of the mice treated with RT only. Each curve represent one individual mouse. FIG. 14C shows the percent difference in tumor volume (the average of w²1/2 and 1²w/2) normalized to the original tumor volume on the day of irradiation, of the mice under conditions: treated with BaTiO₃ and RT; treated with RT only; or No Treatment. FIG. 14D shows survival plot of mice under conditions: treated with BaTiO₃ and RT; treated with RT only; or No Treatment. The ‘death’ in the survival curve is defined as doubling of tumor volume on the day of irradiation (shown as a large square at ≥100% in FIGS. 14A-14C)

FIGS. 15A-15D demonstrate that the BaTiO₃ does not affect mice weight and thus is safe for treatment. FIG. 15A shows weight data of mice treated with BaTiO₃. FIG. 15B shows weight data of mice without being treated by BaTiO₃. FIG. 15C shows the comparison of weight data of mice with and without being treated by BaTiO₃. FIG. 15D shows the comparison of weight data of mice with and without being treated by BaTiO₃, with weight data adjusted to same starting weight.

### DETAILED DESCRIPTION

Before the disclosed processes and devices are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

Radiosensitizer compounds may be used when administering radiation to produce more effective treatments, e.g., by making DNA more susceptible to radiation, or extending the life of free radicals produced by the radiation. Radiation enhancers include elements or compounds that interact directly with the radiation to cause more tissue damage by increasing the absorption or scattering of the radiation, causing more local energy deposition by production of secondary electrons, alpha particles, Auger electrons, ionizations, fluorescent photons, and free radicals. For cancer therapy, the purpose of radiation enhancement is to selectively increase the effective dose to the tumor. Accordingly, radiation enhancers, so including various drugs, elements or compounds generally preferentially accumulate in tumor tissue and/or the tumor tissue must respond in a preferential way, to spare the normal tissue.

This disclosure provides a nanoparticle technology for cancer therapy that can be used in conjunction with other technologies to improve cancer therapy.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclature used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

### Definitions

The term “tumor,” as used herein, refers to an abnormal mass of tissue that results when cells divide more than they should or do not die when they should. In the context of the present disclosure, the term tumor may refer to tumor cells and tumor-associated stromal cells or tissue (i.e., the tumor “microenvironment”). Tumors may be benign and non-cancerous if they do not invade nearby tissue or spread to other parts of the organism. In contrast, the terms “cancerous tumor,” “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The cancerous tumor may be a carcinoma (cancer arising from epithelial cells), a sarcoma (cancer arising from bone and soft tissues), a lymphoma (cancer arising from lymphocytes), a blood cancer (e.g., myeloma or leukemia), a melanoma, or brain and spinal cord tumors. The cancerous tumor can be located in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. More particularly, cancers of the digestive system can affect the esophagus, stomach, small intestine, colon, rectum, anus, liver, gall bladder, and pancreas. Cancers of the respiratory system can affect the larynx, lung, and bronchus and include, for example, non-small cell lung carcinoma. Cancers of the reproductive system can affect the uterine cervix, uterine corpus, ovaries, vulva, vagina, prostate, testis, and penis. Cancers of the urinary system can affect the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). In one embodiment, the cancerous tumor is a colorectal cancer or carcinoma (CRC), a breast cancer, or a melanoma.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

### Methods

In an aspect, disclosed is a method of eliminating tissue or cells by delivery of an agent such as barium (Ba) nanoparticles (NPs) to the tissues or cells, then applying external energy that interacts with the barium nanoparticles. In an embodiment, the method includes the delivery of the agent to blood of a subject for uptake by tissues or cells of the subject. In certain embodiments, the agent is administered with intravenous, intratumoral, peri-tumoral, intraperitoneal, parenteral, intraprostatic, intrapancreatic, intravesical, intrapulmonary, intramammary, or intracolonic injection, or by any other suitable route, such as through the urinary tract, rectum, or via vaginal or uterine delivery. In certain embodiments, the external energy is X-ray, laser light, microwave, particle beam, gamma ray, or ultrasound.

In an aspect, provided is a method for a targeted cancer therapy, said method comprising administering to a subject such as a mammal diagnosed with a cancer, an effective amount of an agent such as a barium nanoparticles or a composition including barium nanoparticles and subjecting said subject to a radiation therapy or ultrasound therapy. In an embodiment, the agent is administered with intravenous injection. In other embodiments, the agent is wherein the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route In an embodiment, the subject is a mammal. In an embodiment, the mammal is a human. In an embodiment, the mammal is a non-human mammal. In an embodiment, the non-human mammal is a cat or a dog. In an embodiment, the subject is a human.

In an aspect, provided is a method for enhancing the effects of radiation directed to a tissue or a population of cells in a subject such as a mammal, the method including administering a therapeutically effective amount of an agent such as barium nanoparticles to said subject. In an embodiment, the method includes achieving a concentration in said tissue or said population of cells of the subject of at least about 0.1% metal by weight, may be less than 0.1% metal by weight, or up to about 10% metal by weight of the subject; and subsequently irradiating the subject with radiation directed to said tissue or said population of cells, wherein said radiation is in the form of X-ray, laser light, microwave, particle beam, or gamma ray. In an embodiment, the particle beam is proton beam. In an embodiment, the power of the radiation is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 keV to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 MeV. In an embodiment, the agent is administered with intravenous injection. In certain embodiments, the agent is wherein the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In an embodiment, the subject is a mammal. In an embodiment, the mammal is a human. In an embodiment, the mammal is a non-human mammal. In an embodiment, the non-human mammal is a cat or a dog. In an embodiment, the subject is a human.

The present invention also provides methods for enhancing the effects of radiation directed to a tissue or cells in or from a subject such as a mammal by administering an effective amount of barium nanoparticles to the subject or to the tissue or cells ex vivo, then irradiating the subject with radiation directed to the tissue or cells, or irradiating the tissue or cells ex vivo. The methods of the present invention are useful for enhancing or improving the ablation of unwanted tissues in a subject such as a mammal without unacceptable damage to surrounding normal tissues or substantial toxicity to the subject. In an embodiment, the subject is a mammal. In an embodiment, the mammal is a human. In an embodiment, the mammal is a non-human mammal. In an embodiment, the non-human mammal is a cat or a dog. In an embodiment, the subject is a human.

In an aspect, the present invention provides methods of enhanced radiation therapy or ultrasound therapy for promoting the shrinkage and/or elimination of tissues targeted for destruction by using barium nanoparticles.

Any suitable barium NPs can be used for this invention, some nonlimiting examples are commercially available BaTiO₃ NPs such as VIVOVIST™ (Nanoprobes, Inc, Yaphank, NY). In an embodiment, the method includes the commercially available BaTiO₃ NP, VIVOVIST™. A suitable product can include about 1 mg/ml to about 9 g/ml, optionally about 300 mg/ml BaTiO₃ NP, but less concentrated or more concentrated compositions may be used. In an embodiment, the method further includes other radiation enhancer or sensitizer, including but not limited to, Fluorouracil. In an embodiment, the method includes a dose (volume) of the barium nanoparticles that depends on tumor size. In an embodiment, the dose of the barium nanoparticles are about 0.001 grams (g) to about 3 g per kilograms (kg). For example, the dose of the barium nanoparticles per kilogram of tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g. In an embodiment, the method includes providing and administering the agent intravenously (IV administration). In some embodiments, the agent is wherein the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route. In an embodiment, the agent is administered with intravenous injection. In an embodiment, the amount of agent administered intravenously per kilogram of the tumor is about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, or about 20.0 g to about 30.0 g. In an embodiment, the agent is administered with intratumoral injection. In an embodiment, the amount of agent administered intratumorally per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

In some embodiments, the size of a barium nanoparticles of the present disclosure is about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm. In some embodiments, the average size and polydispersity of nanoparticles are measured by dynamic light scattering. In one embodiment, the size of the nanoparticles is measured by electron microscopy.

In some embodiments, the barium nanoparticles of the present disclosure further comprise one or more targeting moieties. The targeting moiety targets the barium nanoparticles for delivery to a specific site, a tissue, or subcellular compartment in a subject, e.g., tumor. In some embodiments, the targeting moiety is capable of binding to specific cells, such as cancer cells. In one embodiment, the targeting moiety is capable of facilitating the internalization of the nanoparticles to specific cells, such as cancer cells. In some embodiments, the targeting moiety is a small molecule, antibody, nucleic acid, or peptides.

In some embodiments, the barium nanoparticles of the present disclosure have a crystalline structure that provides a piezoelectric response. In some embodiments, the piezoelectric response of the barium nanoparticles can enhance ultrasound.

The methods may be performed ex vivo or in vivo. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. When the cell is contacted with the composition in vivo, the composition may be administered to an animal, such as a mammal, particularly a human, using standard administration techniques and routes, such as those described herein. In some embodiments, the disclosed methods promote inhibition of tumor cell proliferation, progression, the eradication of tumor cells, and/or a reduction in the size of at least one tumor such that a mammal (e.g., a human, a cat, or a dog) is treated for cancer. “Treatment of cancer” means alleviation of cancer in whole or in part, or inhibit the progression of cancer in whole or in part. In an embodiment, the disclosed methods and compositions reduce the size of a cancerous tumor by at least about 20% (e.g., at least about 25%, 30%, 35%, 40%, 45%, 50%, 55%, 60%, 65%, 70%, 75%, 80%, 85%, 90%, or 95%). In an embodiment, the disclosed methods and compositions inhibit the progression of tumor.

Ideally, the methods described above result in the treatment of the cancerous tumor. As used herein, the terms “treatment” and “treating” can include reversing, alleviating, inhibiting the progression of, preventing or reducing the likelihood of a cancer regrowth, or one or more symptoms or manifestations of a cancer.

In an embodiment, the barium nanoparticles are administered as a part of a surgery, an immunotherapy, an intratumoral cancer therapy, a gene therapy, chemotherapy, a radiation therapy, a targeted therapy, a hormone therapy, a cryoablation, a radiofrequency ablation, a photodynamic therapy, a laser therapy, ultrasound therapy, or any combination thereof. In an embodiment, the agent is administered as a part of solo therapy or a combination therapy.

After reaching the cancer targets, the barium nanoparticles significantly enhance conventional treatment modalities at the cellular level. When an external irradiation source, like X-ray, strikes the GNPs, radicals are generated to induce DNA damage. Due to synergistic multi-level targeting, the therapy can be effective in treating all cancers that utilize radiation therapy such as breast cancer, prostate cancer, colon cancer, brain cancer, or any combination thereof with minimal in vivo side-effects to the normal tissue.

### Compositions

In an aspect, the present disclosure provides a pharmaceutical composition including barium nanoparticles. In an embodiment, the composition includes at least one pharmaceutically acceptable vehicle and/or excipient.

The compositions of the present disclosure can be formulated into any of many possible dosage forms such as, but not limited to, tablets, capsules, gel capsules, liquid syrups, soft gels, suppositories, emulsions or microemulsions, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers. The pharmaceutical composition of the present disclosure may also include a pharmaceutical carrier or excipient. A pharmaceutical carrier or excipient is a pharmaceutically acceptable solvent, suspending agent or any other pharmacologically inert vehicle for delivering one or more cargoes such as nucleic acids to a subject.

In an embodiment, the methods and compositions disclosed herein slow or delay or stop the progression or growth of the tumor. In an embodiment, the methods and compositions disclosed herein slow the progression or growth of the tumor. In an embodiment, the methods and compositions disclosed herein delay the progression or growth of the tumor. In an embodiment, the methods and compositions disclosed herein stop the progression or growth of the tumor.

The present disclosure is illustrated and further described in more detail with reference to the following non-limiting examples.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, “optional” or “optionally” means that the subsequently described event or circumstance may or may not occur, and that the description includes instances where said event or circumstance occurs and instances where it does not.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

A significant change is any detectable change that is statistically significant in a standard parametric test of statistical significance such as Student's t-test, where p<0.05.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### EXAMPLES

### Example 1: Loading of Human Prostate Cancer Cells (DU145) and Mouse Pancreatic Cancer Cells (PANC02) with BaTiO₃ Nanoparticles In Vitro

Methods: DU145 human prostate cancer cells and PANC02 mouse pancreatic cancer cells were grown in the presence of 4 mM to 12 mM BaTiO₃ Nanoparticles (NPs) for three days. The cells were washed to remove unassociated NPs, removed with trypsin and analyzed by flow cytometry.

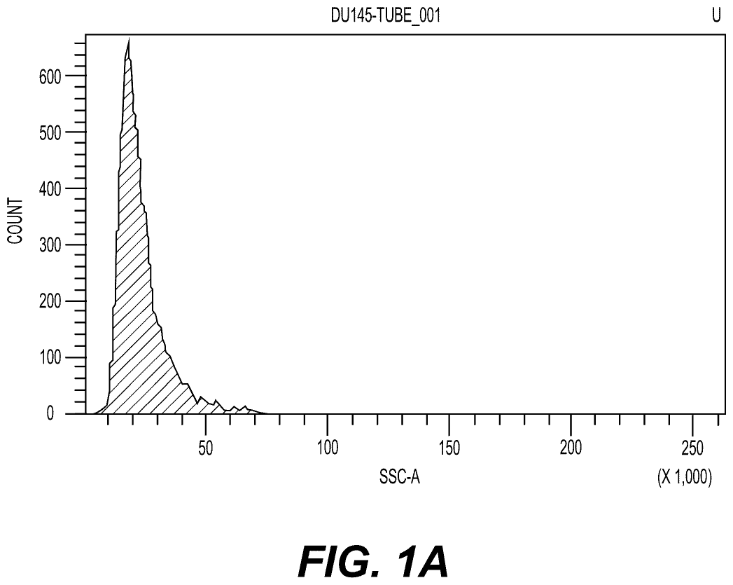

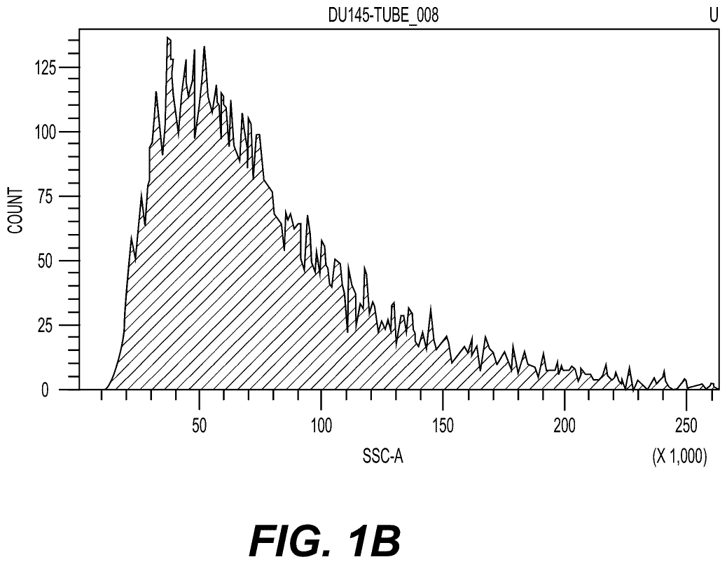

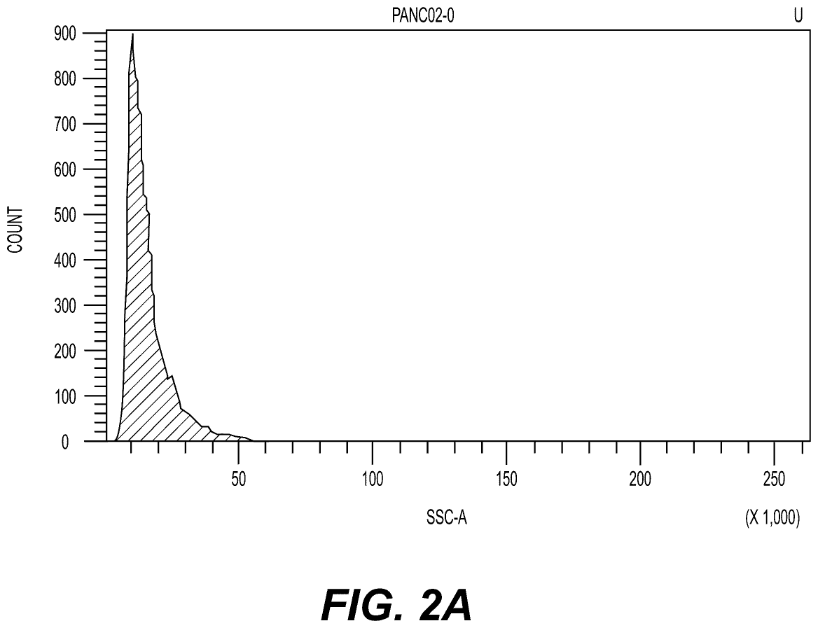

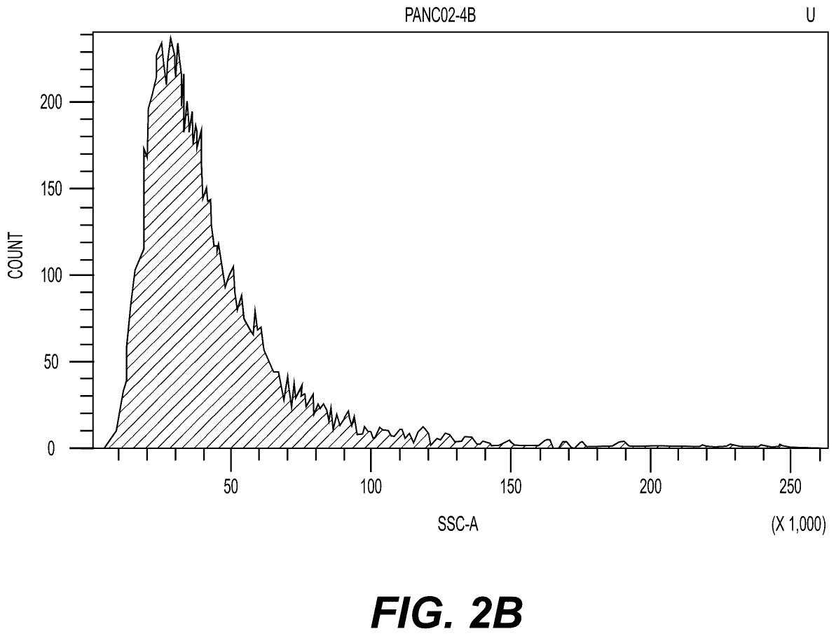

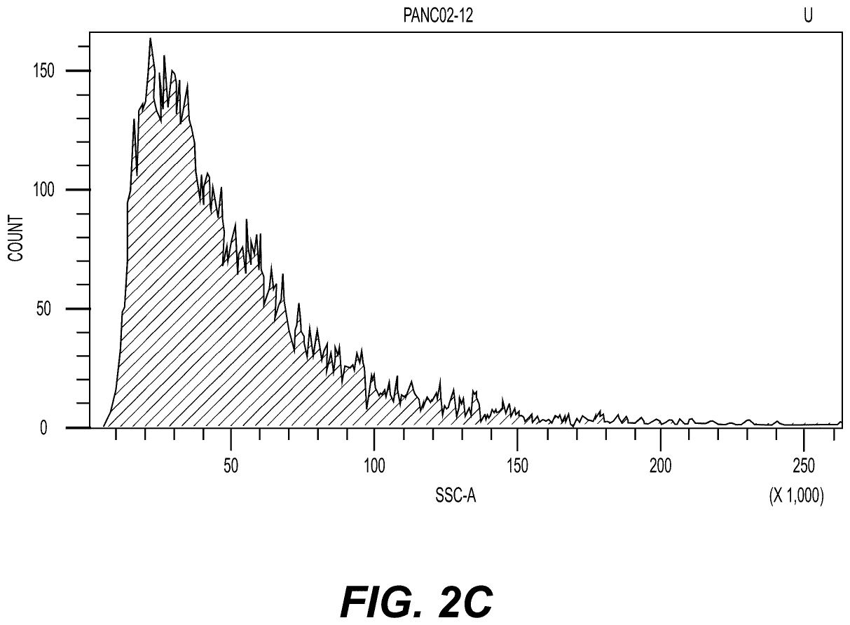

Results: For DU145 cells, side scatter is shown to increase about 4-fold from about 20,000 (untreated cells) to about 80,000 (treated cells). For PANC02 cells, side scatter is shown to increase about 2.5-fold from about 15,000 to about 40,000 (FIGS. 1A-1B and FIGS. 2A-2C).

These experiments show that incubation of tumor cells with BaTiO₃ NPs results in the tight association of the NPs with the cells. The NPs cannot be washed away by repeated rinses. Further, the NPs, by virtue of their light scattering ability, alter side scatter measurements made by flow cytometry.

### Example 2: BaTiO₃ Nanoparticles are Taken Up into Prostate and Pancreatic Cancer Cells

Methods: DU145 human prostate cancer cells and PANC02 mouse pancreatic cancer cells were incubated with BaTiO₃ Nanoparticles (4 mM) in vitro for three days. The cells were washed repeatedly, pelleted, fixed with glutaraldehyde in cacodylate buffer and processed for electron microscopy.

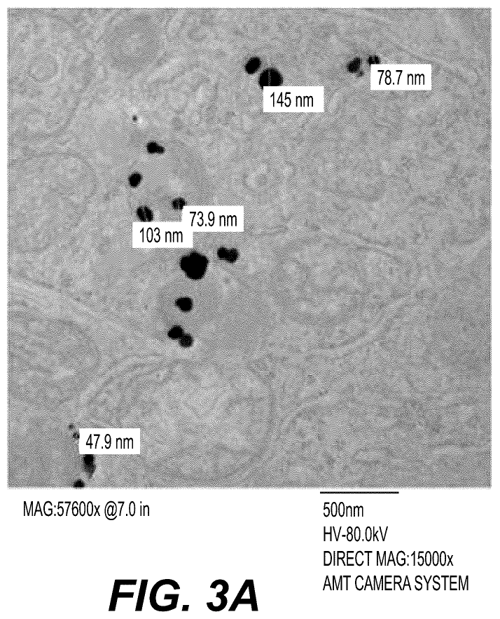

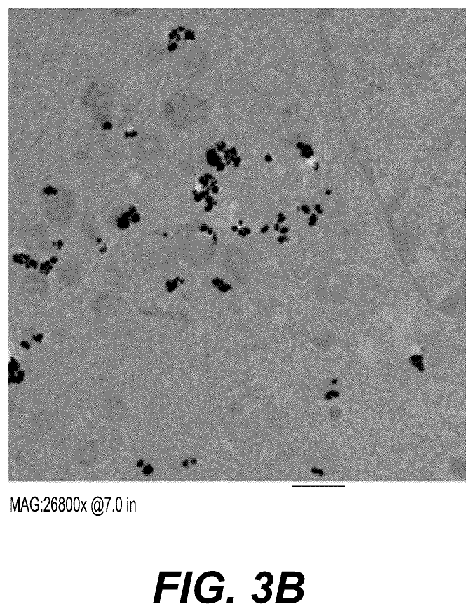

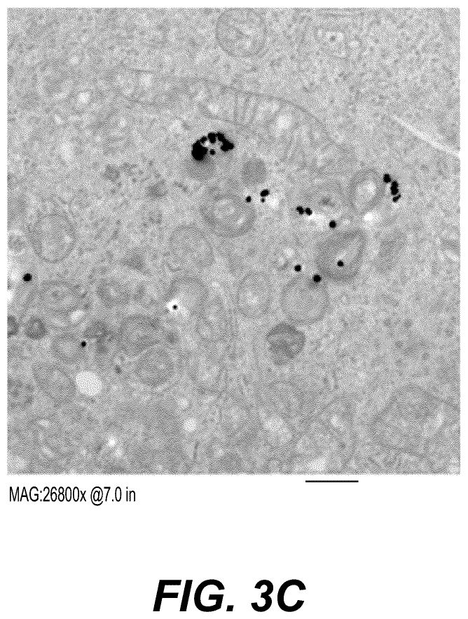

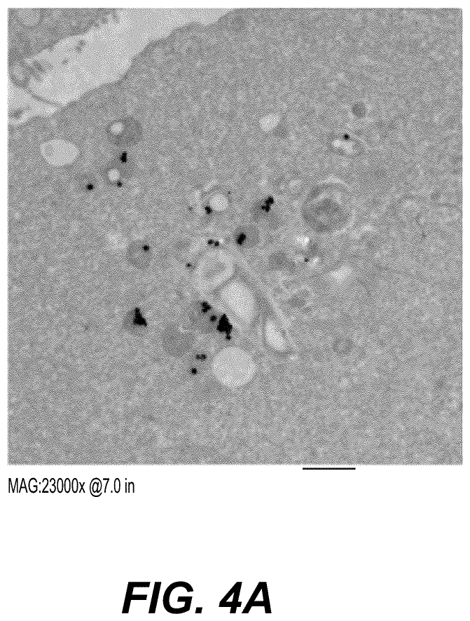

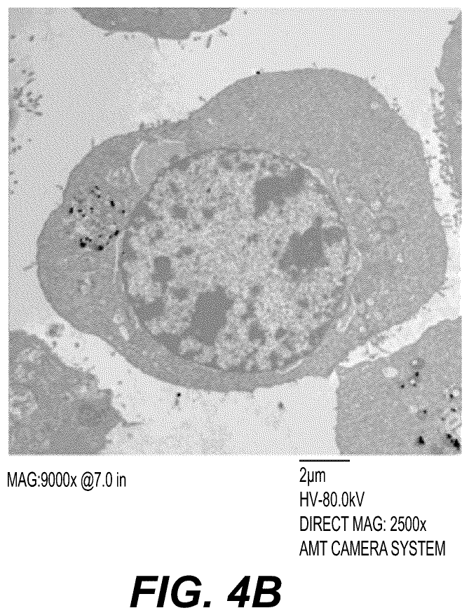

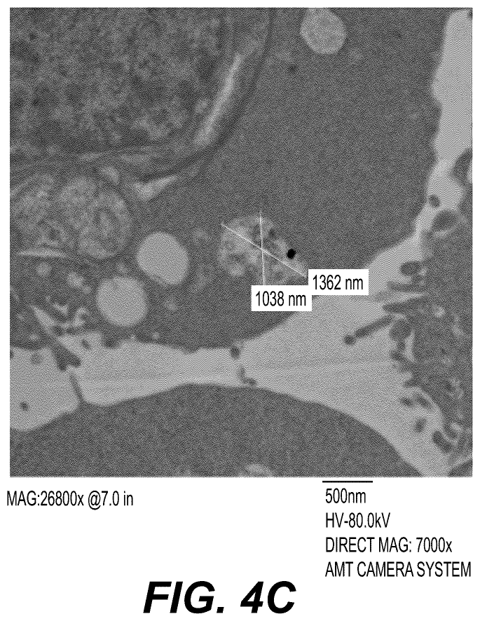

Results: As shown in FIGS. 3A-3C and FIGS. 4A-4C, the electron Microscopy provides proof that BaTiO₃ NPs are taken up into DU145 human prostate cancer cells and PANC02 mouse pancreatic cancer cells after incubation of the cells with the NPs in vitro. BaTiO₃ NPs are black spheres that measure about 70 nm. Dimers measure about 150 nm. NPs are seen in cell organelles outside of the nucleus. This illustrates that intracellular BaTiO₃ NPs have the potential to enhance the effects of radiation therapy.

### Example 3: Viability Assay Shows that DU145 Human Prostate Cancer Cells Loaded with BaTiO₃ NPs Die More after 6 MV Irradiation than Untreated Cells

Methods: DU145 human prostate cancer cells were either untreated or loaded with BaTiO₃ NPs in vitro. Cells from both groups were either unirradiated or irradiated with 8 Gy using a 6 MeV hospital irradiator. Cells (1000) from each group were plated into wells (96 well plate; 8 wells/group) and allowed to grow for 3 days. Viable cells in the wells were assayed using the XTT viability assay according to manufacturer specifications.

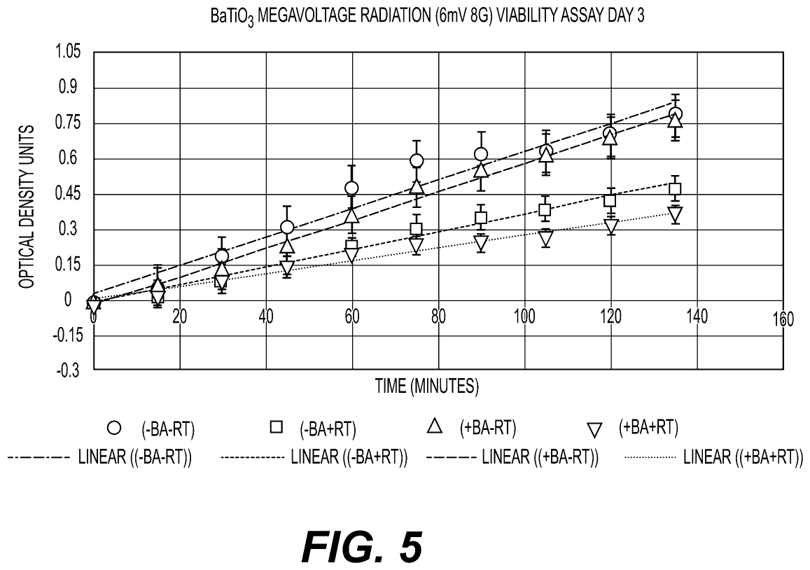

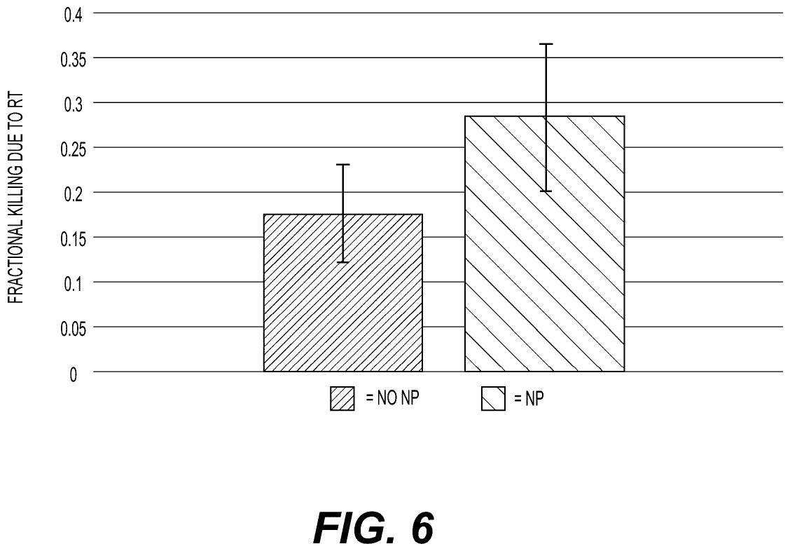

Results: As shown in FIG. 5 and FIG. 6, the uptake of BaTiO₃ NPs into human prostate cancer cells increases tumor cell killing after radiation therapy (RT) resulting in a decreased amount of viable tumor cells in culture 3 days after RT. In these experiments, the amount of BaTiO₃ loading into the tumor cells allows RT to be about 1.5-fold more effective.

A BaTiO₃ dose enhancement of about 1.3-fold to 1.6-fold was observed repeatedly in four experiments. The bar graph shows the results of another one of these experiments with statistical analysis (FIG. 6).

### Example 4: Loading of DU145 Human Prostate Cancer Cells with BaTiO₃ Nanoparticles (NPs) Increases Cell Killing after Radiation Therapy (RT) as Shown by the Clonogenic Assay

Methods: Du145 human prostate cancer cells were either untreated or loaded with BaTiO₃ NPs. Cells from both groups were either unirradiated or irradiated with 4 or 8 Gy using the UCHC 6 MV irradiator. Cells in each group were diluted so that each 100 ul aliquot contained 1 cell (96 wells plated). The wells were assayed for the presence or absence of a colony after 3 weeks. The total number of colonies was also recorded.

Results: The data show that cells loaded with BaTiO₃ NPs formed fewer colonies after 6 MV irradiation. Dose enhancement factor (DEF) was calculated as the ratio of fractional inhibitions obtained. Surprisingly, inventors have found that BaTiO₃ NPs increased effective RT dose about 15-fold after 8 Gy RT and about 1.5-fold after 4 Gy RT. Both the XTT viability assay and the clonogenic assays have shown qualitatively the same effect, providing strong evidence that prior loading of human prostate cancer cells with BaTiO₃ NPs significantly enhances clinically-relevant RT of the tumor cells (Table 1).

TABLE 1
BaTiO₃ Clonogenic Assay

| Experiment | Conditions | No Ba No RT | No Ba + RT | Ba No RT | Ba + RT | DEF |
|---|---|---|---|---|---|---|
| 1 | 4 mM, 6 mV, 8 Gy | 37/96 wells | 7/96 wells | 32/96 wells | 1/96 wells | |
| | Jun. 11, 2024 | 45 colonies | | 40 colonies | | |
| 2 | 12 mM, 6 mV, 8 Gy | 36/96 wells | 6/96 wells | 53/96 wells | 0/96 wells | |
| | Jun. 21, 2024 | 46 colonies | | 77 colonies | | |
| 1&2 | Total | 73/192 wells | 13/192 wells | 85/192 wells | 1/192 wells | 15.2 |
| | | 91 colonies | | 117 colonies | | |
| 3 | 12 mM, 6 mV, 4 Gy | 46/96 wells | 12/96 wells | 44/96 wells | 8/96 wells | |
| | Jul. 9, 2024 | 52 colonies | | 52 colonies | | |
| 4 | 12 mM, 6 mV, 4 Gy | 43/96 wells | 14/60 wells | 40/96 wells | 7/60 wells | |
| | Jul. 11, 2024 | 56 colonies | | 56 colonies | | |
| 3&4 | Total | 89/192 wells | 26/156 wells | 84/192 wells | 15/156 | 1.63 |
| | | 108 colonies | | 108 colonies | | |

### Example 5: BaTiO₃ NPs are Tolerated Well after IV Injection

Methods: Out bred mice were injected with BaTiO₃ NPs at 3.5 g/kg. The mice were weighed and observed over time.

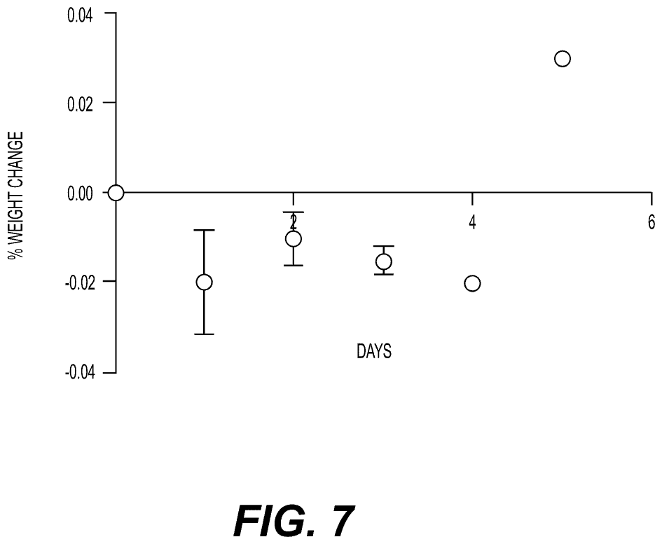

Results: As shown in FIG. 7, the mice lost about 2% of their weight the day after injection at 3 g/kg, but then gained weight normally. After 6 days their weights were indistinguishable from that of control mice. The mice did not show signs of distress or illness over the course of a year.

This experiment demonstrated that clinically relevant amounts of the BaTiO₃ NPs appear to be tolerated well by mice after IV injection.

### Example 6: Loading of PANC02 Tumors with BaTiO₃ NPs In Vivo

Methods: Five million Mouse PANC02 pancreatic tumor cells were injected subcutaneously into the thighs of immunocompromised mice. When the tumors were advanced, BaTiO₃ NPs (3 g/kg) were injected into the mice intravenously. MicroCT imaging of tumors was performed 24 hours post IV injection.

Results: As shown in FIG. 8, high levels of contrast are seen in the tumors compared to the surrounding soft tissues of the thigh. Contrast was distributed heterogeneously in the tumor. Quantification of contrast using Ba standards revealed a range of uptake with regions of very high contrast containing about 20% Ba injected dose/gram tumor and regions of low contrast containing 1-3% Ba injected dose/gram tumor.

Such levels of BaTiO₃ tumor loading might be expected to provide dose enhancements of 2-fold to −10-fold. Such dose enhancements would be expected to provide clinical benefit.

### Example 7: Microlocalization of BaTiO₃ Nanoparticles in PANC02 Tumors

Methods: BaTiO₃ NPs that have been taken up into PANC02 tumors in vivo can be visualized by darkfield microscopy by virtue of their ability to scatter light. Darkfield microscopy provides excellent resolution of BaTiO₃ NPs that have been taken up into PANC02 tumors in vivo. Twenty-four hours after IV injection of BaTiO₃ NPs, the mice were euthanized and the legs containing the tumors were fixed in 4% buffered formalin overnight. The legs were then transferred into 30% sucrose for 2-3 days to cryopreserve the tissue. Tumors were then removed from the legs, placed into cassettes containing cryomatrix and rapidly frozen. Cryosections (7 micron) were then subjected to darkfield microscopy.

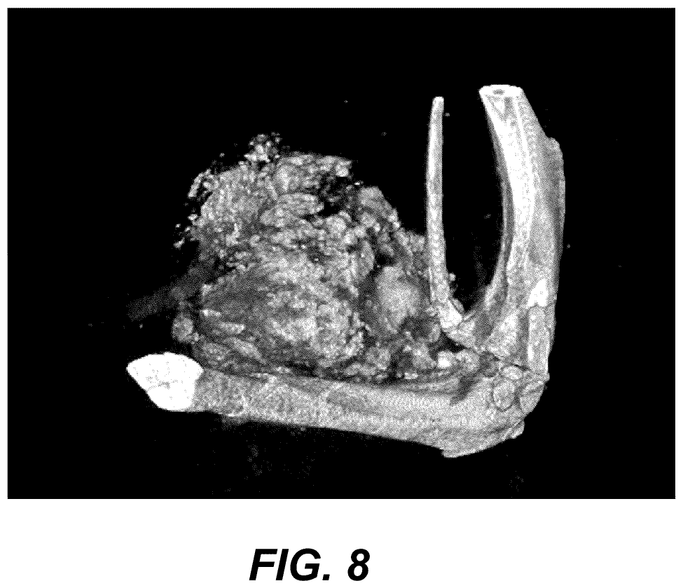

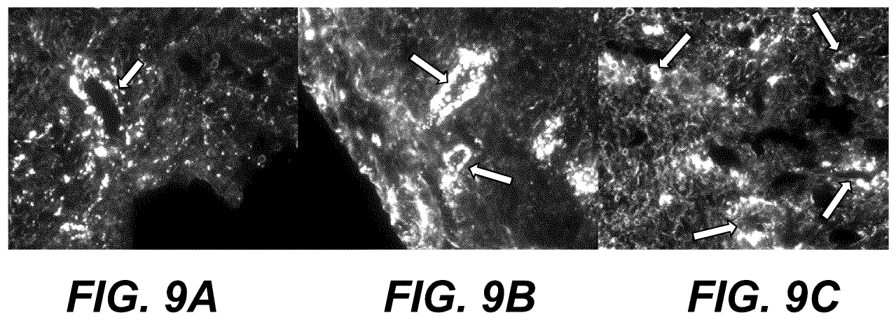

Results: As shown in FIGS. 9A-9C BaTiO₃ contrast is seen as white. BaTiO₃ NPs are seen to be more prevalent on the periphery of the tumor (growing edge) than the tumor interior. BaTiO₃ NPs are seen to be preferentially associated with tumor vessel-like structures (arrows). The results show increased RT damage to tumor vessels, especially those located in the growing edge of the tumor, would be expected to have a deleterious effect on the growth of the tumor.

### Example 8: VE Cadherin Immunofluorescence in PANC02 Tumors

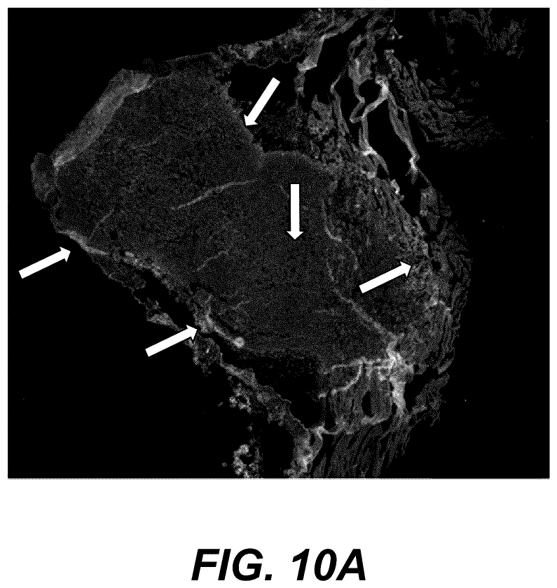

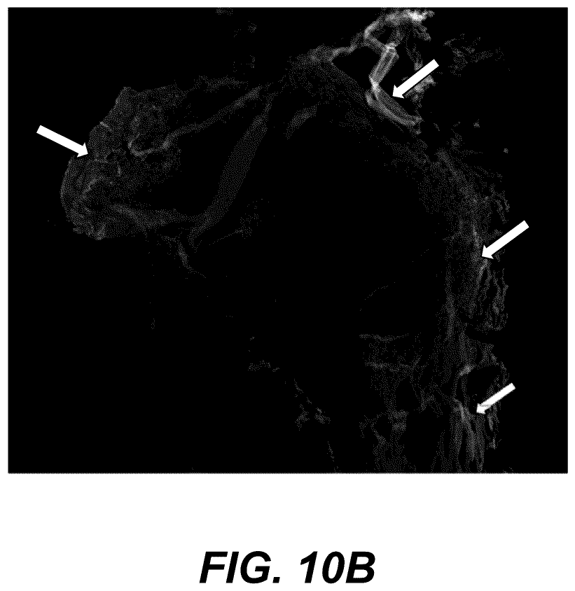

Methods: Cryosections of PANC02 tumors containing BaTiO₃ NPs were subjected to immunofluorescence using a primary antibody to VE Cadherin and a fluorescent secondary antibody (488 nm, green). The FIG. 10A represents a tissue section treated with anti-rabbit VE Cadherin followed by 488—labeled goat anti-rabbit antibody. The FIG. 10B represents a tissue section treated with just 488-labeled goat anti-rabbit antibody.

Results: As shown in FIGS. 10A-10B, while there is some non-specific auto-fluorescence seen (white arrows, FIG. 10B), specific VE Cadherin fluorescence is seen throughout (White arrows, FIG. 10A). Most of the VE Cadherin immunofluorescence is on the tumor periphery where most of the BaTiO₃ NP contrast is also seen (Not shown).

VE Cadherin is known to be associated with endothelial cells in tumor vessels as well as with abnormal vesicular structures called vasculogenic mimicry found in advanced tumors. The specific immune fluorescence seen here shows that it will be possible to visualize the relationship between the localization of VE Cadherin and the BaTiO₃ NPs.

### Example 9: BaTiO₃ Nanoparticles (NPs) Colocalize with Tumoral VE Cadherin

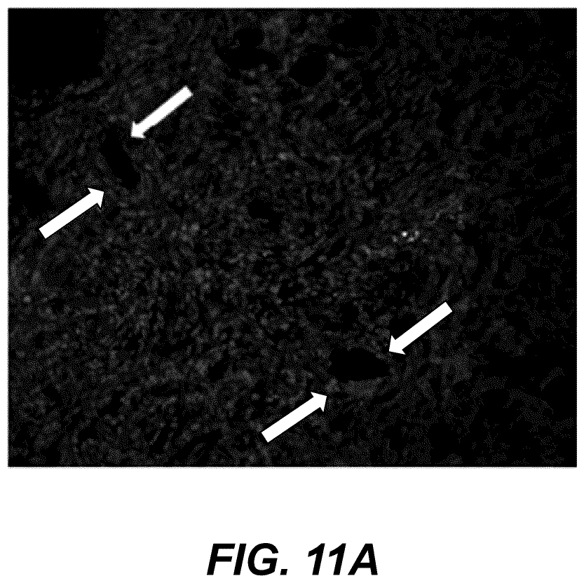

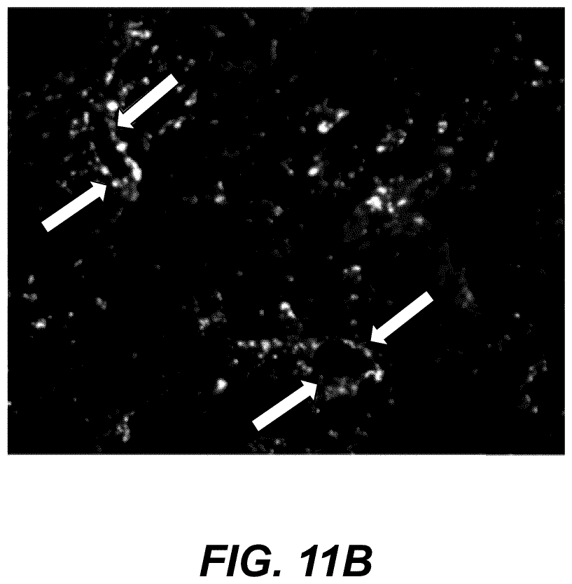

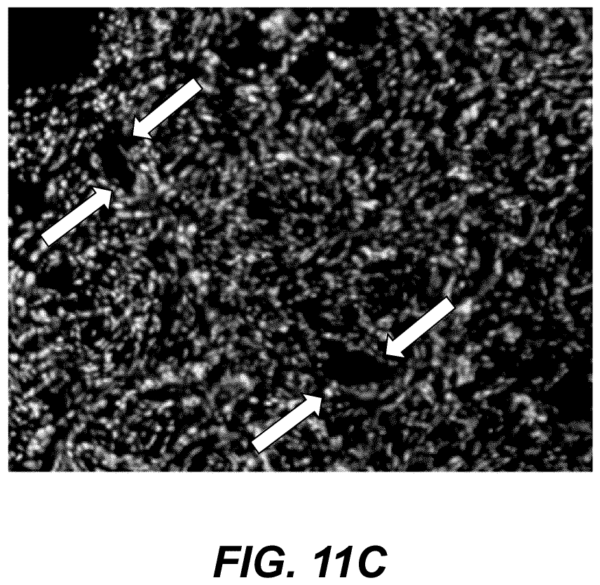

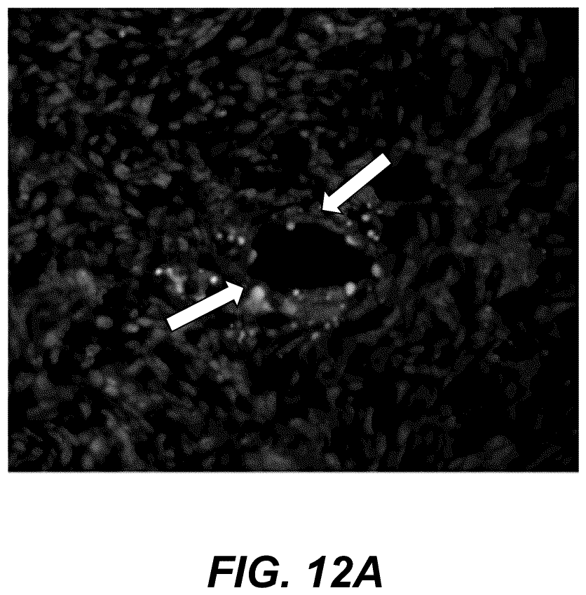

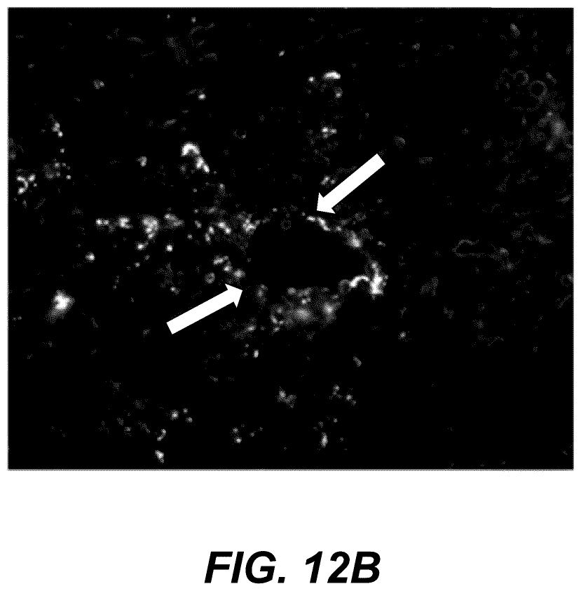

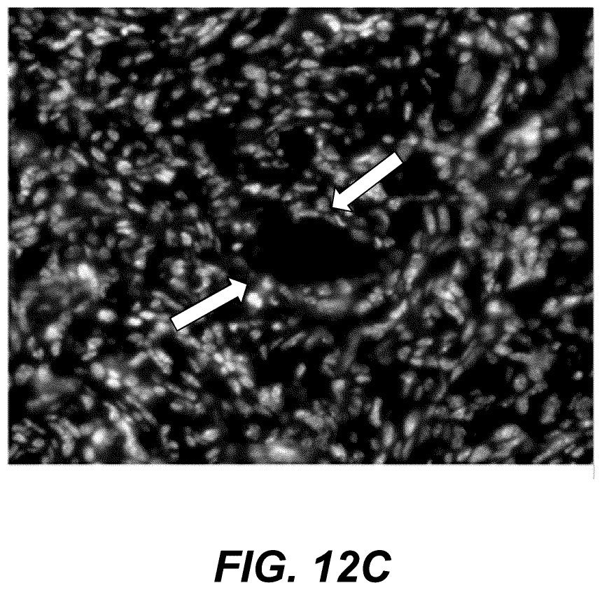

Methods: Shown are two higher power (20X (FIGS. 11A-11C) and 40X (FIGS. 12A-12C)) images of tumor regions showing both BaTiO₃ localization as seen by darkfield microscopy (white, FIG. 11B and FIG. 12B) and the same region showing specific VE Cadherin fluorescent staining (white, FIG. 11A and FIG. 12A), and Nuclei (white, FIG. 11C and FIG. 12C).

Results: All of the intense white representing BaTiO₃ NPs (FIG. 11B and FIG. 12B) colocalizes with white representing VE Cadherin fluorescence (FIG. 11A and FIG. 12A). There appears to be VE Cadherin fluorescence that lacks intense white stain. More work is needed to determine whether there are regions with VE Cadherin stain that lack BaTiO₃ NPs i.e. just how much of the two molecules overlap in the tumor and precisely what structures are being labeled by either stain.

VE Cadherin localizes to both normal and abnormal tumor vessels. BaTiO₃ NPs preferentially load PANC02 tumors. Since BaTiO₃ NPs colocalize with VE Cadherin in the PANC02 tumors, the NPs therefore localize to normal and abnormal vessels in the tumor. Since the BaTiO₃ NPs have been shown to significantly enhance RT of 6 MV radiation, they will enhance RT dose to tumor vessels, particularly in the tumor growing edge. This should have a significant detrimental effect on tumor growth as tumors are highly dependent on their vessels for growth.

### Example 10: Colocalization of BaTiO₃ NPs, CD133 (Tumor Stem Cells) and CD31 (Endothelial Cells) in Panc02 Cells

Methods: Cryosections of Panc02 tumors previously labeled with BaTiO₃ NPs were labeled with CD133 (tumor stem cell marker) and CD31 (endothelial cell marker). For CD31 labeling, the primary antibody used is goat anti CD31 polyclonal antibody and the secondary antibody used is 647 Donkey anti-Goat antibody. For CD133 labeling, the primary antibody is Rabbit anti-CD133 polyclonal antibody, and the secondary antibody is 488 Goat anti-Rabbit antibody.

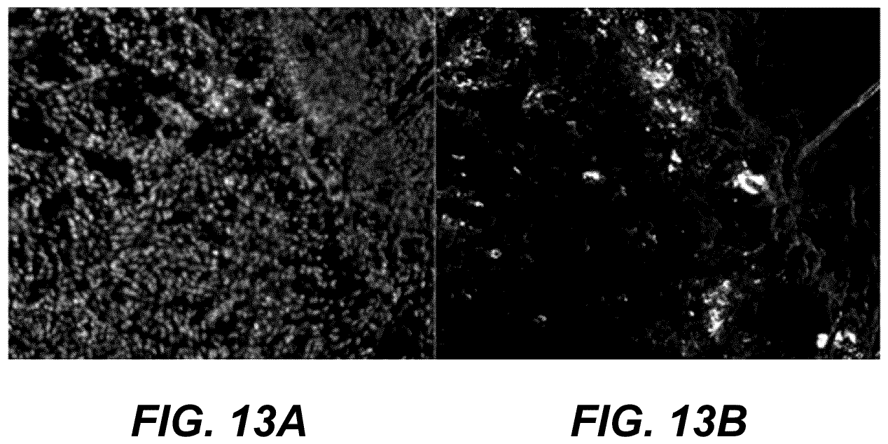

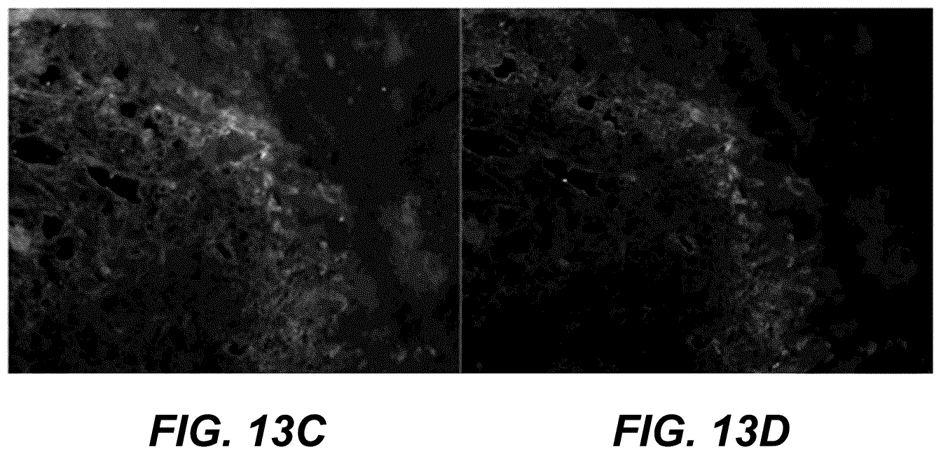

Results: As shown in FIGS. 13A-13D BaTiO₃ (white, FIG. 13B), CD31 (white, FIG. 13C), CD133 (white, FIG. 13D), specific staining is greatest along the growing edge of the tumor. There is considerable overlap between BaTiO₃ (white, FIG. 13B), CD31 (white, FIG. 13C), and CD133 (white, FIG. 13D) stains. The white stain in FIG. 13A indicates nuclei.

The data shows that BaTiO₃ NPs colocalize with both a blood vessel (CD31, endothelial cell) marker and a tumor stem cell marker (CD133), mostly at the tumor growing edge. Increased radiation dose to blood vessels and tumor stem cells in the tumor growing edge would be expected to be deleterious to tumor growth.

BaTiO₃ NPs are well tolerated nanoparticles that are taken up by human prostate tumor cells growing in vitro when the NPs are added to the cell culture medium. Prostate tumor cells that have accumulated the BaTiO₃ NPs die significantly more than untreated tumor cells after irradiation at a hospital megavoltage irradiator.

Intravenously injected BaTiO₃ NPs also are taken up in large amounts by mouse pancreatic tumors growing subcutaneously on the mouse thigh. The NPs are found in largest amounts in the growing tumor edge of the tumor localized to vessel structures that colocalize with VE Cadherin and CD31 and likely represent both normal and abnormal tumor vessels. They also colocalize with CD133, which is a marker for tumor stem cells.

It has been hypothesized that TiO₂ NPs enhance megavoltage RT by increasing free radical production upon irradiation. The TiO₂ NPs are too toxic to use in vivo but the well tolerated BaTiO₃ NPs can be injected safely into the mouse blood in clinically relevant amounts. Results disclosed herein demonstrate that the BaTiO₃ NPs are suitable for use in the clinic to enhance the RT of tumors and to help with the delincation of tumors.

### Example 11: BaTiO₃ Nanoparticles Enhance Radiation Therapy of Subcutaneous (Sc) Panc02 Tumors Growing in Athymic Nude Mice

Method: Panc02 tumor cells were injected subcutaneously in athymic nude (neu-/neu-) mice. Mice were paired into two groups of approximately equal tumor size. Ten days after implantation, one group received BaTiO₃ NPs (3 g/kg). Approximately 30 hours later, the tumors were irradiated (about 15 Gy). Radiation dose was confined to a 1 cm diameter radiation field encompassing the tumor excluding the mouse body. Briefly the mouse was placed in a Lexan box with a 9 mm Lexan lid. The radiation field was centered on the tumor and a about 5 mm thick slab of tissue-equivalent material (Superflab) was overlayed on the mouse leg. The nominal dose rate was 1400 MU/min (about 10 Gy/minute at depth of maximal dose (about 1.6 cm for the beam energy used) for a 10 cm×10 cm field size, defined at a distance of 100 cm from the target of the machine. Tumors were measured using calipers.

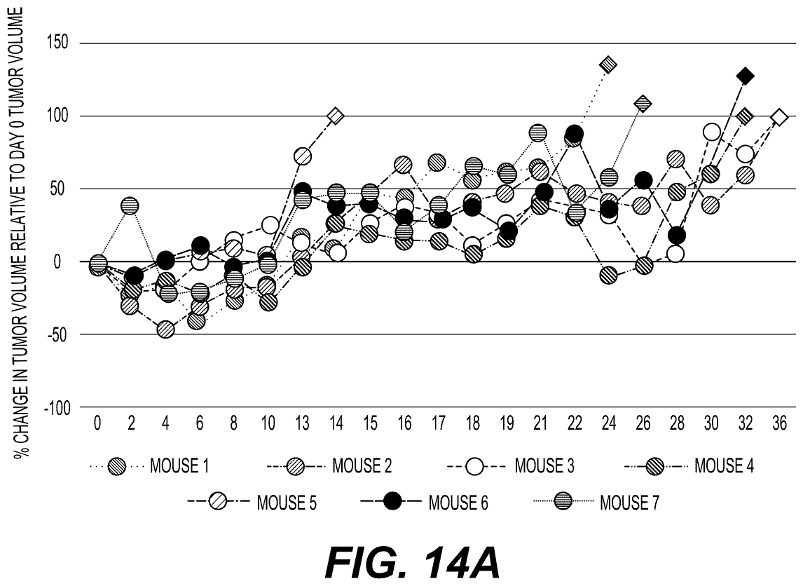

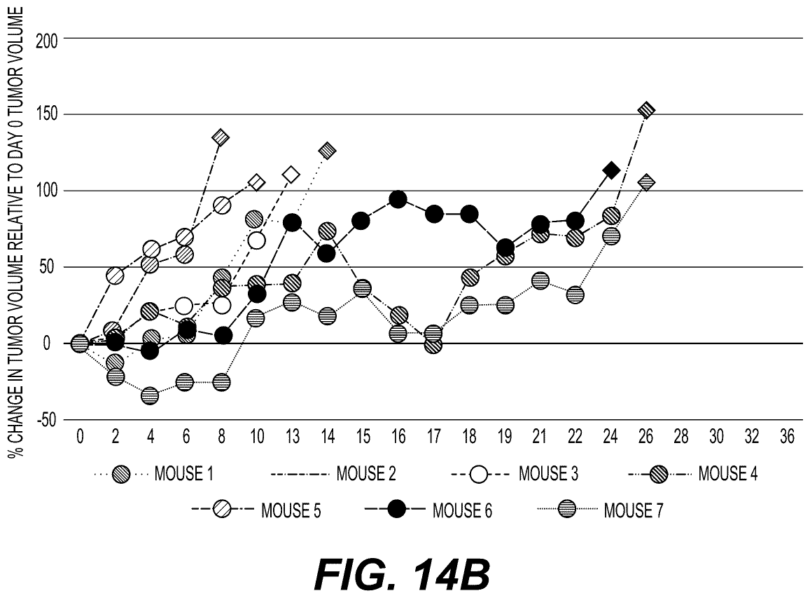

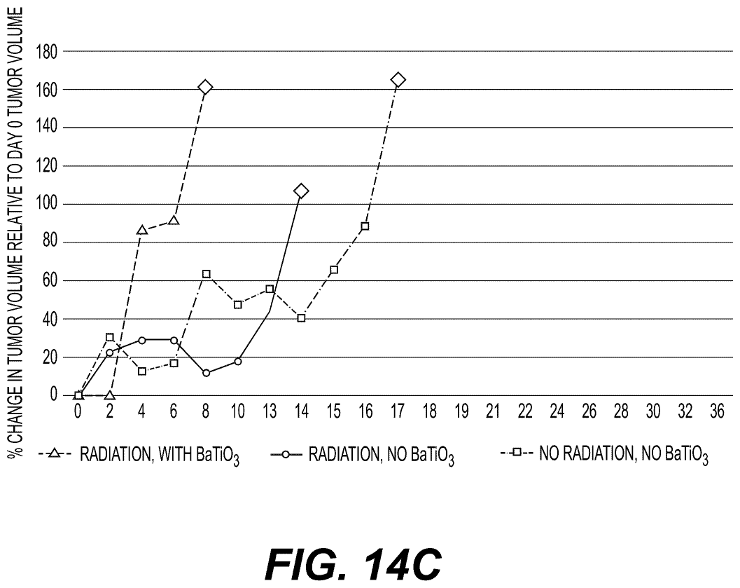

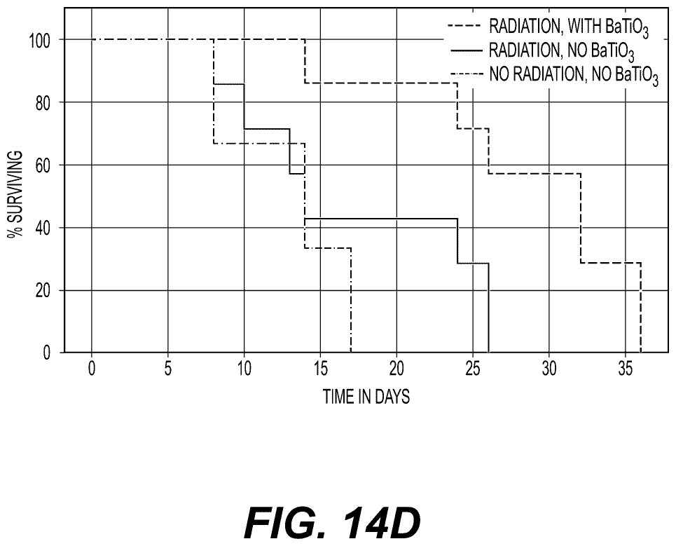

Results: FIGS. 14A-14B show the percent difference in tumor volume (the average of w²1/2 and 1²w/2) normalized to the original tumor volume on the day of irradiation. The tumor volumes ranged from 68 mm³ to 214 mm³; Group A (BaTiO₃+RT), average tumor size was 136 mm³ and median tumor size was 124 mm³; Group B (RT only), average tumor size was 117 mm³ and median tumor size was 128 mm³. Mice weight was also measured during the experiment to evaluate the safety of the nanoparticles.

FIG. 14C shows the percent difference in tumor volume (the average of w²1/2 and 1²w1/2) normalized to the original tumor volume on the day of irradiation, of the mice under conditions: (A) treated with BaTiO₃ and RT; (B) treated with RT only; and (C) No Treatment. Each color represents a different mouse (N=7 per group). Negative values represent tumor shrinkage; positive values represent tumor increase. On each day for which data was available, the percent difference in tumor volumes was rank ordered with the largest negative value being 1, the largest positive value being 14. Wilcoxon non-parametric statistics for small groups was used to analyze statistical significances: Day 4, p<0.1; Day 6, p<0.05; Day 8, p<0.1; Day 10, p<0.01; Day 13, p<0.05; Day 14, p<0.05; Day 15, p>0.1. There was a statistically significant difference in tumor volume increase/decrease between groups A and B on days 6, 10, 13 and 14 post RT. The data was also analyzed using a ‘survival plot’ (FIG. 14D). We defined the doubling of tumor volume on the day of irradiation (shown as a large square at ≥100% in FIGS. 14A-14C) as ‘death’. The ‘survival plot’ shown in FIG. 14D shows that median day of ‘death’ has been extended by more than 50% by the use of the BaTiO₃ NPs and p=0.05 (Log-Rank Test). FIG. 14B shows that tumors in mice that did not receive the BaTiO₃ NPs prior to RT shrunk less and grew faster than tumors that had taken up the BaTiO₃ NPs prior to RT (FIG. 14A). This result supports our hypothesis that BaTiO₃ NPs given IV 24 hrs prior to RT, enhances RT efficacy of sc Panc02 tumors. Strategies to optimize BaTiO₃ uptake into tumors should increase BaTiO₃ efficacy further.

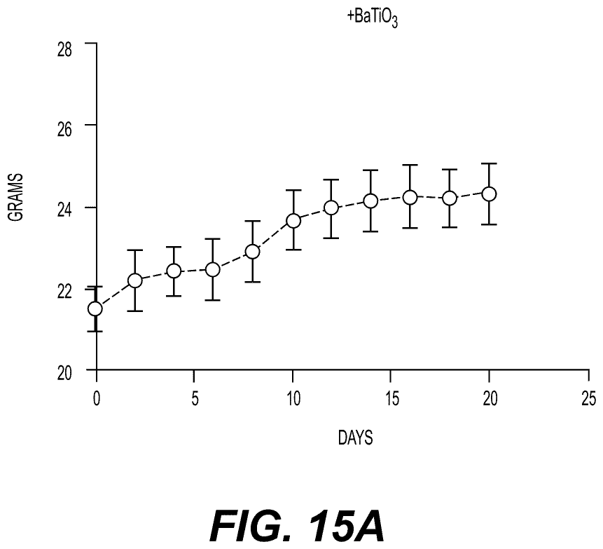

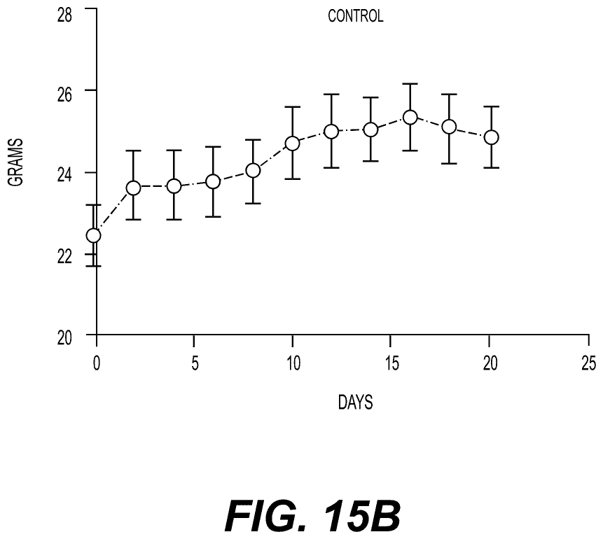

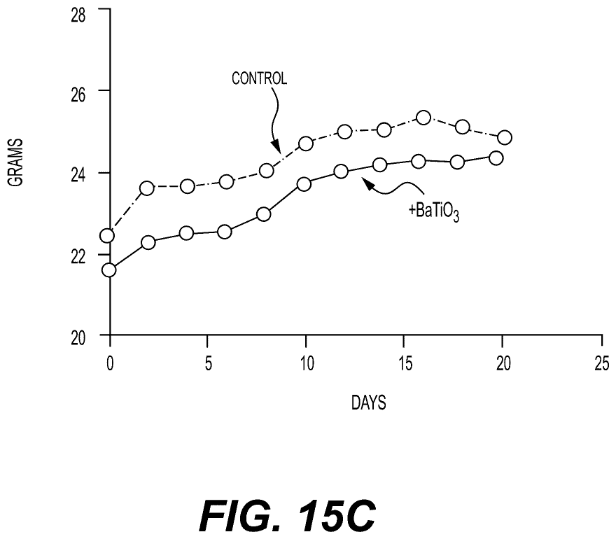

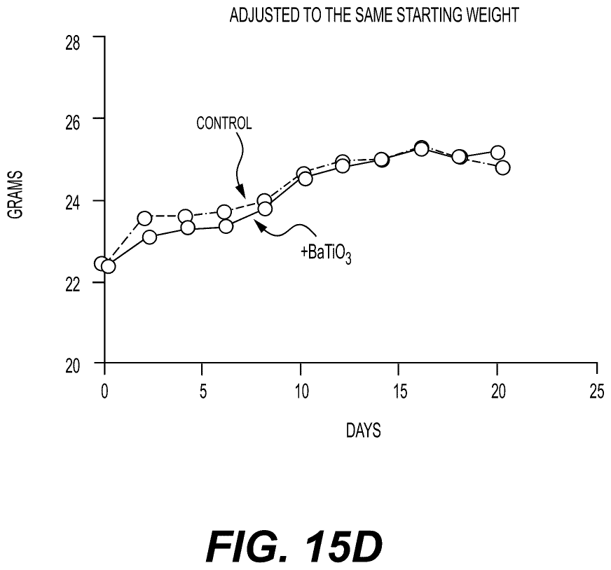

The weight of mice shows that BaTiO₃ NPs has high safety and low toxicity. FIG. 15A shows weight of mice treated with BaTiO₃ NPs (n=10), and FIG. 15B shows weight of mice not treated with BaTiO₃ NPs (n=9). Both groups show a similar trend of weight increase over the 20 days experiment (FIG. 15C). The weights of both groups were adjusted to the same starting weight and it is shown that there is no difference in weight change between the two groups, proving that the BaTiO₃ NPs demonstrate high safety for treatment (FIG. 15D).

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of enhancing effects of radiation directed to a tissue or a population of cells of a subject in need thereof, comprising:
administering an effective amount of barium nanoparticles to the subject, such that said barium nanoparticles are brought into the vicinity of or into contact with said tissue or a population of cells; and
subjecting the subject to radiation under the presence of said barium nanoparticles.

2. The method of claim 1, wherein the subject is mammal.

3. (canceled)

4. The method of claim 1, wherein said tissue or said population of cells is tumor.

5. (canceled)

6. The method of claim 4, wherein the solid tumor is selected from the group comprising of prostate cancer, pancreatic cancer, lung cancer, breast cancer, colon cancer, carcinoma, melanoma, sarcomas, lymphoma, and any combination thereof.

7. The method of claim 1, wherein the barium nanoparticles are BaTiO₃ nanoparticles.

8. The method of claim 1, wherein the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

9. The method of claim 1, wherein the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route.

10-13. (canceled)

14. The method of claim 1, wherein the effective amount of barium nanoparticles per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

15. The method of claim 1, wherein the radiation is X-ray, laser light, microwave, particle beam, or gamma ray.

16-18. (canceled)

19. The method of claim 15, wherein the radiation is X-ray, and wherein the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 keV to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 MeV.

20-40. (canceled)

41. A method of treating cancer in a subject in need thereof, the method comprising:
administering an effective amount of barium nanoparticles or a composition including barium nanoparticles to the subject; and
administering radiotherapy to the said subject.

42. The method of claim 41, wherein the subject is mammal.

43. (canceled)

44. The method of claim 41, wherein the cancer is selected from the group comprising of prostate cancer, pancreatic cancer, lung cancer, breast cancer, colon cancer, carcinoma, melanoma, sarcomas, lymphoma, and any combination thereof.

45. The method of claim 41, wherein the barium nanoparticles are BaTiO₃ nanoparticle.

46. The method of claim 41, wherein the size of the barium nanoparticles are about 1 nm to about 2 nm, about 2 nm to about 3 nm, about 3 nm to about 4 nm, about 4 nm to about 5 nm, about 5 nm to about 10 nm, about 10 nm to about 15 nm, about 15 nm to about 20 nm, about 20 nm to about 25 nm, about 25 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 nm to about 500 nm.

47. The method of claim 41, wherein the barium nanoparticles are administered with intravenous, intratumoral, peri-tumoral, intraperitoneal, parenteral, intraprostatic, intrapancreatic, intravesical, intrapulmonary, intramammary, or intracolonic injection, or by any other suitable route.

48-51. (canceled)

52. The method of claim 41, wherein the effective amount of barium nanoparticles per kilogram of the tumor is about 0.001 g to about 0.002 g, about 0.002 g to about 0.003 g, about 0.003 g to about 0.004 g, about 0.004 g to about 0.005 g, about 0.005 g to about 0.01 g, about 0.01 g to about 0.02 g, about 0.02 g to about 0.03 g, about 0.03 g to about 0.04 g, about 0.04 g to about 0.05 g, about 0.05 g to about 0.1 g, about 0.1 g to about 0.2 g, about 0.2 g to about 0.3 g, about 0.3 g to about 0.4 g, about 0.4 g to about 0.5 g, about 0.5 g to about 1.0 g, about 1.0 g to about 2.0 g, about 2.0 g to about 3.0 g, about 3.0 g to about 4.0 g, about 4.0 g to about 5.0 g, about 5.0 g to about 6.0 g, about 6.0 g to about 7.0 g, about 7.0 g to about 8.0 g, about 8.0 g to about 9.0 g, about 9.0 g to about 10.0 g, about 10.0 g to about 20.0 g, about 20.0 g to about 30.0 g, about 30.0 g to about 40.0 g, about 40.0 g to about 50.0 g, about 50.0 g to about 60.0 g, about 60.0 g to about 70.0 g, about 70.0 g to about 80.0 g, about 80.0 g to about 90.0 g, or about 90.0 g to about 100.0 g.

53. The method of claim 41, wherein radiation of the radiotherapy is X-ray, laser light, microwave, particle beam, or gamma ray.

54-56. (canceled)

57. The method of claim 41, wherein the radiation is X-ray, and wherein the X-ray is about 1 keV to about 2 keV, about 2 keV to about 4 keV, about 4 keV to about 6 keV, about 6 keV to about 8 keV, about 8 keV to about 10 keV, about 10 keV to about 20 keV, about 20 keV to about 40 keV, about 40 keV to about 60 keV, about 60 keV to about 80 keV, about 80 keV to about 100 keV, about 100 keV to about 1000 keV, about 1000 keV to about 5000 keV, about 5000 keV to about 10,000 keV, about 10,000 keV to about 25,000 keV, about 25,000 keV to about 50,000 keV, about 50,000 keV to about 75,000 keV, about 75,000 keV to about 100,000 keV, about 100,000 keV to about 200,000 keV, about 200,000 keV to about 300,000 keV, about 300,000 keV to about 400,000 keV, about 400,000 keV to about 500,000 keV, about 500,000 keV to about 600,000 keV, about 600,000 keV to about 700,000 keV, about 700,000 keV to about 800,000 keV, about 800,000 keV to about 900,000 keV, about 1 MeV to about 2 MeV, about 2 MeV to about 3 MeV, about 3 MeV to about 4 MeV, about 4 MeV to about 5 MeV, about 5 MeV to about 6 MeV, about 6 MeV to about 7 MeV, about 7 MeV to about 8 MeV, about 8 MeV to about 9 MeV, about 9 MeV to about 10 MeV, about 10 MeV to about 15 MeV, about 15 MeV to about 20 MeV, about 20 MeV to about 25 MeV, about 25 MeV to about 30 MeV, about 30 MeV to about 35 MeV, about 35 MeV to about 40 MeV, about 40 MeV to about 45 MeV, about 45 MeV to about 50 MeV, or about 50 MeV to about 100 MeV.

58-61. (canceled)

62. A pharmaceutical composition comprising:
(1) an effective amount of barium nanoparticles; and
(2) at least one pharmaceutical carrier and/or excipient.

63-97. (canceled)
